(19) United States
(12) Patent Application Publication
(10) Pub. No.: US 2019/0336332 A1
(43) Pub. Date: Nov. 7, 2019

(54) TREATMENT OF SPINAL MUSCULAR ATROPHY BY INDUCING HEAT SHOCK RESPONSE

(71) Applicant: The Research Institute at Nationwide Children's Hospital, Columbus, OH (US)

(72) Inventors: DAWN S. CHANDLER, Bexley, OH (US); Catherine E. Dominguez, Baltimore, MD (US)

(21) Appl. No.: 16/310,084

(22) PCT Filed: Jun. 15, 2017
(86) PCT No.: PCT/US17/37601

§ 371 Date: Dec. 14, 2018

Related U.S. Application Data
(60) Provisional application No. 62/350,347, filed on Jun. 15, 2016.

(51) Int. Cl. A61F 7/12; A61K 31/133; A61K 31/616

(52) U.S. Cl. CPC A61F 7/12; A61F 2007/126; A61K 31/616; A61K 31/133

(57) ABSTRACT

A method of treating spinal muscular atrophy by inducing a heat shock response in a subject in need thereof is described. The heat shock response can be induced by heating the temperature of a tissue region of the subject above 37° C., or by administering a therapeutically effective amount of a heat shock inducing agent.

### CONTINUING APPLICATION DATA

This application claims the benefit of U.S. Provisional Application Ser. No. 62/350,347 filed Jun. 15, 2016, which is incorporated by reference herein.

### STATEMENT REGARDING FEDERALLY SPONSORED RESEARCH

This invention was made with government support under grant numbers R21-NS084187 and F31-NS079032 awarded by the National Institute of Neurological Diseases and Stroke. The Government has certain rights in this invention.

### BACKGROUND

Spinal muscular atrophy (SMA) is a neurodegenerative disease and is one of the greatest sources of genetic mortality in infants with an incidence of approximately 1 in 6,000 births. Low levels of the protein, SMN, cause degeneration of alpha motor neurons, which leads to muscle wasting. Over 90% of SMA cases are characterized as severe, which result in respiratory distress and often death within 2 years. SMN protein is encoded by two genes, SMN1 and SMN2. These two genes are nearly identical, but a key difference is a translationally silent single nucleotide switch in exon 7. This difference causes SMN2 to mis-splice and prevents exon 7 inclusion in the majority of its transcripts during RNA splicing.

SMA patients have inherited deletions or mutations of SMN1, leaving only SMN2 and its largely mis-spliced transcripts responsible for generating all SMN protein. Full-length SMN2 transcripts do give rise to identical proteins as SMN1; however, transcripts lacking exon 7 (Δ7) produce truncated, unstable proteins, which are non-functional and often rapidly degraded. Therefore, the lack of SMN1 is not fully compensated for by SMN2.

While no therapy currently exists, efforts to combat SMA revolve around stabilizing the SMN protein, increasing transcription rate or stability of SMN2 mRNA transcripts, or altering splicing of SMN2.

### SUMMARY OF THE INVENTION

The invention provides a method for modulating SMN2 splicing in a therapeutic context for spinal muscular atrophy with heat shock response. The therapeutic heat shock response can be achieved using a heat shock inducing agent or heat shock inducing condition. The heat shock response-inducing compounds include protein synthesis inhibitors, proteasome inhibitors, serine protease inhibitors, Hsp90 inhibitors, NSAIDS, and hydroxylamine derivatives. Additionally, a co-inducer such as sodium salicylate or arimoclomol can be used. Conditions for inducing a heat shock response include heating tissue of the subject to a temperature above 37° C. Examples of means that can be used to induce a heat shock response include photoelectric, mechanical, chemical, and temperature devices.

In one aspect, the present invention provides a method of treating spinal muscular atrophy (SMA) that includes the step of inducing a heat shock response in a subject in need thereof. In some embodiments, the heat shock response is induced by heating the temperature of a tissue region of the subject above 37° C. In further embodiments, the heat shock response is induced by administering a therapeutically effective amount of a heat shock inducing agent.

In various embodiments, administration of the heat shock inducing agent increases full length SMN2 mRNA and full length SMN2 protein in neural cells, muscle cells, or glial cells of the subject by at least about 50%. In various further embodiments, the heat shock inducing agent can be a protein synthesis inhibitor, a proteasome inhibitor, a serine protease inhibitor, an Hsp90 inhibitor, a non-steroidal anti-inflammatory drug, or a hydroxylamine derivative. In some embodiments, the heat shock inducing agent is a co-inducer (e.g., arimoclomol).

The heat shock inducing agent can be administered in a variety of different ways. In some embodiments, the heat shock inducing agent is administered into the central nervous system. In other embodiments, the heat shock inducing agent is administered into the cerebrospinal fluid. In further embodiments, the head shock inducing agent is administered into muscle. In yet further embodiments, the heat shock inducing agent is administered in drinking water.

In some embodiments, the subject in need of treatment is a human. In further embodiments, the subject is a fetus and the heat shock response is induced in utero. The subject in need of treatment may be a subject that has been diagnosed with type I SMA, type II SMA, type III SMA, or type IV SMA.

### BRIEF DESCRIPTION OF THE FIGURES

FIG. 1 provides a schematic representation of the SMN1 and SMN2 genes, the RNA transcribed from this DNA, and the protein products produced as a result of translation of the RNA.

FIGS. 2A-2C provide graphs and images showing that heat shock increases the functional splicing product of SMN2 in a dose and time-dependent manner. A. Neuroblastoma cell line, SHSY-5Y, were differentiated for 7-9 days in 5 μM retinoic acid and then treated with various stresses to characterize the level of SMN2 as a stress-responsive gene. RNA splicing levels of SMN1 and 2 were observed by performing RTPCR analysis on total mRNA harvested from cell lysates. To mimic oxidative stress, cells were treated with peroxide treatment for 24 hours, heat shock was achieved with 41° C. treatment for 24 hours, and hypoxia was performed with 1% O₂ for 48 hours, B. To test the cell-type specificity of the heat shock effect on SMN splicing, we performed a heat shock treatment on several cell lines. All treatments were performed with 24 hours of 41° C. and compared with NT (37° C.) quantitation is shown (n=3). This treatment was performed in Differentiated SHSY-5Y cells, SMA patient fibroblasts (GM03813), SMA carrier fibroblasts (GM03814), and a breast cancer cell line (MCF-7) cells. Heat treatment induces a significant change in splicing in all cell lines tested. C. To test the duration of heat treatment necessary for heat shock induced splicing correction a time course was performed. Differentiated SHSY-5Y cells were treated with 41° C. for 1, 4, 8, and 24 hour time intervals. D. To test the quantity of heat required to elicit splicing correction, a heat treatment dose response was performed. Differentiated SHSY-5Y cells were treated with temperatures from 37° to 41° C. for 8 hours. Significance levels are: * pvalue<0.05, ** p-value <0.01.

FIGS. 3A and 3B provide graphs and images showing that correction of SMN2 splicing correlates with increases in SMN protein expression and positive splicing factors. A. Differentiated SHSY-5Y cells and SMA patient fibroblasts exposed for 24 hrs to 37 or 41° C. heat treatment. Whole cell lysates were harvested in RIPA buffer and separated with SDS-PAGE. Through Western blot analysis, SMN protein levels were analyzed and used actin levels as a loading control. Quantitation of three biological replicates is shown below the respective cell type. B. Quantitative PCR was performed from RNA isolated from 24 hr heat treated SHSY5Y cells compared to NT controls. Two technical replicates of three biological replicates are shown. Significance was determined with t-test. * p-value<0.05.

FIG. 4 provides a scheme, image, and graph showing that Tra2Beta binding sites are critical for the heat-induced SMN splicing correction. Minigene mutational analysis of Tra2b binding sites was performed on SMN minigenes comprised of exons 6, 7, and 8, with a truncated intron 6 and a full sized intron 7. Mutations were induced in SMN minigenes by site-directed mutagenesis. Two separate mutations were performed for the splicing enhancer region, SE2, which TRA2Beta is known to bind. SE2b mutation corresponds to the mutation of the first three nucleotides of the TRA2Beta binding site, and SE2c mutation corresponding to the mutation of the last three nucleotides of the TRA2Beta binding site. Minigenes were then transfected into differentiated SH-SY5Y cells, which were subsequently treated with 37° or 41° for 24 hrs. Following treatment, total RNA was harvested from cells and minigenes were PCR-amplified using a minigene-specific tag, to prevent interference from endogenous SMN transcripts. Significance levels are: * p-value<0.05

FIG. 5 provides images and a graph showing that Tra2Beta is necessary for the SMN splicing correction under heat shock. siRNA-mediated knock downs were performed in MCF7 cells. Cells were treated with siRNA duplexes targeting SRSF1 (30 nM siSRSF1) or TRA2Beta (80 nM siTRA2Beta) and a non-specific siRNA (siNS) and compared to untreated cells (NT). Cells were transfected for 24 hours with RNAiMAX transfection reagent followed by 24 hours of either 37° or 41° C. Following treatments, cells were harvested for total RNA or protein. The ability of these siRNA duplexes to knock down their targets was confirmed by Western blot analysis. RT-PCR was performed from RNA harvests to observe SMN exon 7 splicing levels. Graphical representation of SMN exon 7 inclusion is shown and is the product of three biological replicates. Significance levels are: * pvalue<0.05.

FIG. 6 provides images and a graph showing that HSF90 is involved in the heat shock SMN splicing modulation. siRNA-mediated knock down of HSP90 and HSF1 was performed in MCF-7 cells. As before, cells were treated with siRNA duplexes targeting HSP90 (100 nM siHSP90), HSF1 (100 nM siHSF1), or non-specific siRNA sequences (siNS). Cells were transfected for 24 hours with RNAiMax transfection reagents and followed with a heat treatment (37° or 41° C.) for 24 hours. Cells were harvested for total RNA and whole cell lysate for protein. Protein knock down was confirmed by western blot analysis with actin as a loading control. RT-PCR was utilized to amplify SMN transcripts to observe exon 7 splicing under treatments. Graphical representation of SMN exon 7 inclusion is shown and is the product of three biological replicates. Significance levels are: * p-value<0.05.

FIG. 7 provide an image and a graph showing that HSP90 inhibition prevents heat-induced SMN splicing correction. HSP90 inhibition was performed with 17-DMAG in MCF-7 cells. Dose responses were analyzed at 0, 10, 20, 40 nM 17-DMAG for 8 hours, with or without 41° C. heat treatment. Total RNA was harvested and RT-PCR amplification of SMN transcripts was used to observe splicing levels of exon 7. Significance levels are: * p-value<0.05, ** p-value <0.01.

FIGS. 8A-8D provide graphs showing that heat treatment does not improve severe SMA pup disease parameters. A. Taiwanese SMA neonatal pups were treated daily with 45 minutes of 40.5° C. in a thermal chamber. Groups were divided into two cohorts, one for survival studies and one for tissues harvests. Tissues cohorts were sacrificed and tissues collected at PND11, while aging studies were allowed to age indiscriminately. B. Treatment groups of Het and SMA mice were either treated with 40.5° C. or left untreated (NT), and analyzed for improvements in righting reflex at post-natal day 11 to measure muscle function. No significant alterations observed. C. The same treatment groups were analyzed for improvements in body weight over time. Body weights were measured daily until postnatal day 14. D. Kaplan-Meier curve indicating survival times for treatment groups. Significance was determined with log-rank test in Prism6.

FIGS. 9A and 9B provide an image and graph showing that heat treatment does not alter SMN splicing in SMA mice. Heat-treated Taiwanese SMA mice were incubated beginning at PND1 with 40.5° for 45 minutes per day. A. At PND11, tissues (spinal cord, brain, quad, and liver) were isolated and RNA harvested through Trizol extraction. RNA was used for RT-PCR amplification of human SMN2 transgene transcripts. B. Quantitation of SMN2% exon 7 inclusion in heterozygous (n=3), 40.5° SMA (n=3), and untreated (NT) SMA mice (n=7) are depicted. No significant change in splicing was observed.

FIGS. 10A and 10B provide an image and graph showing T-the increase in full-length SMN2 transcript in response to heat-shock treatment persists for at least 8 hours after return to normal temperature. A. MCF-7 cells were seeded at 3×10⁵ cells/well in triplicate in six 6-well plates and incubated at 37° C. for 24 h. Five plates were then treated with a heat-shock of 39° C. for 2 h. RNA samples were collected from one plate immediately after the 2 h heat-shock (time-point 0). The remaining plates were returned to 37° C. and RNA samples were collected from them at the time-points indicated in the graph, as well as from the control plate that received no heat shock. cDNA was synthesized from the RNA samples, followed by RT-PCR amplification using primers to SMN exons 6 and 8. The PCR products were digested with DdeI restriction enzyme to distinguish SMN1 and SMN2 products by size when resolved on an agarose gel. B. The relative quantity of bands representing full-length vs. skipped exon 7 (Δ7) SMN2 transcripts (arrows) was measured using Image Lab 2.0.1 software with images acquired on a Bio-Rad Gel Doc XR. The graphed values are the average of three biological replicates, with error bars representing the standard error of the mean. P-values were calculated in pairwise comparisons using Student's t-test, with values <0.05 considered significant. Asterisks indicate values significantly different from the “no heat shock” control value.

FIGS. 11A-11C provide images and a graph showing siRNA-mediated knock-down of HSF1 (heat-shock factor 1) levels does not affect the increased SMN2 exon 7 inclusion response to heat-shock. A. MCF-7 cells were seeded at 3×10⁵ cells/well in triplicate in four 6-well plates and incubated at 37° C. for 24 h. Cells in two plates were transfected with a non-specific siRNA and in the other two plates with a cocktail of 4 siRNAs to HSF1 using RNAiMAX reagent. After an additional 24 h of incubation at 37° C., one plate of each transfection was shifted to 41° C. for 24 h. RNA and protein (whole-cell lysates) were collected from all samples. Protein samples were resolved by SDS-PAGE, transferred to PVDF membrane and the blot was probed first with an anti-HSF1 primary antibody, and then with an anti-Tra2B antibody. The samples treated with HSF1 siRNA showed a substantial reduction in HSF1 protein relative to the non-specific siRNA-treated samples, while the TRA2B signal was roughly consistent in both sets of samples, demonstrating equivalent loading of protein in each lane. B. RT-PCR using primers to SMN exons 6 and 8 was performed on cDNA synthesized from the RNA samples. The PCR products were digested with DdeI restriction enzyme to distinguish SMN1 and SMN2 transcripts, and resolved on an agarose gel. C. The band intensities for SMN2 full-length and exon 7-skipped (Δ7 SMN2) were measured using Image Lab 2.0.1 software. Graphed values are the average of three biological replicates with error bars representing the standard error of the mean. P-values were calculated in pairwise comparisons using Student's t-test, with values <0.05 considered significant. Asterisks indicate p-values <0.0001 between the control and heat-shocked samples.

### DETAILED DESCRIPTION OF THE INVENTION

The present invention provides a method of treating the neurodegenerative disease spinal muscular atrophy by inducing a heat shock response, which corrects the incorrect splicing of the survival of motor neuron 2 (SMN2) gene and subsequently increases protein levels of its full-length form.

### Definitions

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a”, “an”, and “the” are inclusive of their plural forms, unless contraindicated by the context surrounding such.

The terms “comprising” and variations thereof do not have a limiting meaning where these terms appear in the description and claims.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

“Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc.

“Pharmaceutically acceptable” as used herein means that the compound or composition is suitable for administration to a subject to achieve the treatments described herein, without unduly deleterious side effects in light of the severity of the disease and necessity of the treatment.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses. An effective amount, on the other hand, is an amount sufficient to provide a significant chemical effect, such as the activation of a heat shock response by a detectable amount.

A “peptide” or “polypeptide” is a linked sequence of amino acids and may be natural, synthetic, or a modification or combination of natural and synthetic.

One aspect of the invention provides a method of treating spinal muscular atrophy (SMA) by inducing a heat shock response in a subject in need thereof. As described herein, the inventors have discovered that the heat shock response modulates SMN2 splicing to increase the amount of full length SMN2 mRNA, whose expression increased the amount of full length SMN2 protein. Full length, as used herein, refers to the normal and effective form of the survival neuron protein, or mRNA encoding the same, as opposed to truncated forms of the protein that are expressed from mRNA transcripts lacking exon 7.

Administration of the heat shock inducing agent or providing conditions that induce a heat shock response increase the full length SMN2 mRNA and full length SMN2 protein in cells of the subject. Examples of cells in which SMN2 is affected include neural cells, such as motoneuron cells, glial cells, and muscle cells. The reference level of full length SMN2 mRNA or full length SMN2 protein can be the level in a cell of the subject prior to treatment, or a cell that has not been treated. The method can increase the expression level of full length SMN2 mRNA or full length SMN2 protein by at least about 30%, 50%, 75%, 100%, 150%, 200%, 300%, 400%, 500% or greater. Alternatively, the increase can be measured by the ratio of transcripts containing exon 7 to those lacking exon 7. This ratio can be increased by at least about 30%, 50%, 75%, 100%, 150%, 200%, 300%, 400%, and 500% or greater.

Heat shock inducing agents or conditions increase the amount of full length SMN2 mRNA or SMN2 protein in cells of the subject. The cells can be either in vivo, in vitro, or ex vivo. The cells of the invention are derived from SMA patients. The cells are termed “SMA cells” herein. The cells are isolated from a variety of sources and tissues. For example, the cells can be isolated from cerebrospinal fluid or from a biopsy. Examples of cells include neural cells, a glial cell, or a muscle cell. The cells can be propagated in culture according to cell type and origin of the cells. The cells can be propagated without being immortalized. Alternatively, the cells can be immortalized using a virus or a plasmid bearing an oncogene, or a transforming viral protein, e.g., papilloma E6 or E7 protein.

The “subject” of the invention refers to human or non-human mammal, e.g. a dog, a cat, a mouse, a rat, a cow, a sheep, a pig, a goat, or a primate, and expressly includes laboratory mammals, livestock, and domestic mammals. In one embodiment, the mammal may be a human; in others, the mammal may be a rodent, such as a mouse or a rat. In another embodiment, the subject is an animal model (e.g., a transgenic mouse model) of SMA. Alternatively, the subject is an SMA patient. The SMA patient can be homozygous or heterozygous for mutations in SAM.

Spinal muscular atrophy is a genetically inherited pediatric neurodegenerative disease for which splicing alterations are central to the disease pathology. SMA is characterized by degeneration of alpha spinal motor neurons; a process that leaves target muscles denervated which consequently atrophy due to lack of stimulation. Interestingly, though all cells in an SMA patient exhibit drastically decreased levels of the survival of motor neuron (SMN) protein, only the motor neurons and certain populations of cortical neurons appear to be affected by this deficit (d'Errico et al., PloS One, 8(12), e82654 (2013)). While this disease is genetically straightforward, the cause for the cell-specificity of SMA is less well known. To begin to understand this phenomenon it is important to understand the specific microenvironment of these cell types and to determine the stresses that could lead to cellular exacerbation of the phenotype.

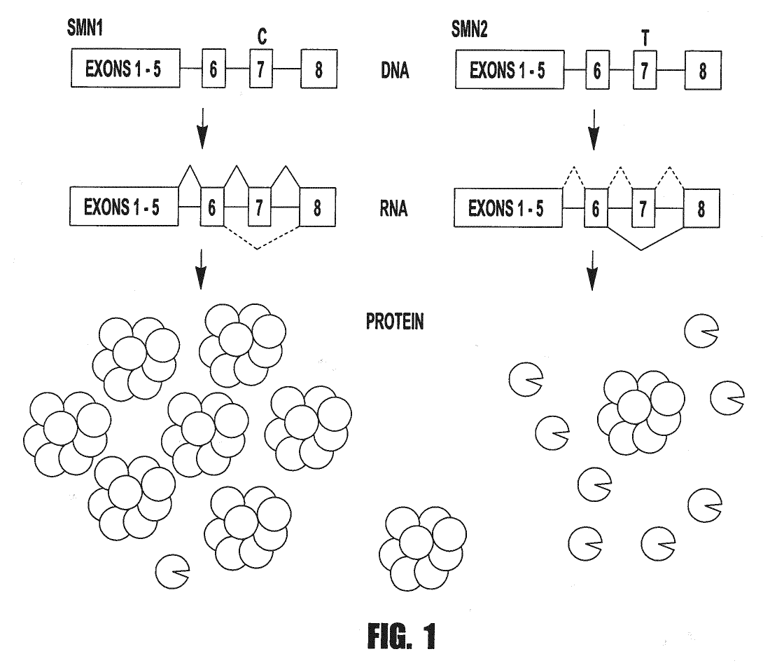

SMN is encoded by two genes in humans: SMN1 and SMN2. The two genes are nearly identical, but a single base alteration within the DNA of exon 7 causes a deviation in the mRNA splicing patterns of the two genes (FIG. 1). mRNA processing requires the mRNA message to be compiled correctly by the spliceosome, which removes introns and selectively chooses which exons should be included in the final message. These decisions are determined by splice site strength, supportive or inhibitive sequences within the transcript, known as enhancers or repressors, respectively, and the availability and activity of protein accessory factors that recognize these sequences. At the DNA level, the 6th nucleotide of exon 7 in SMN1 contains a cytosine, which creates an mRNA splicing enhancer to recruit the spliceosome and help the exon to be recognized. On the other hand, at the same respective position, SMN2 contains a thymine, which alters the enhancer to become a repressor in the resultant mRNA, which blocks the spliceosome from including the exon. Therefore, this single base pair alteration in the DNA of SMN1 and SMN2 creates mRNA sequences with differential baseline splicing responses. While the C to T alteration from SMN1 to SMN2 is translationally silent, the result is an mRNA splicing pattern in which SMN1 transcripts contain exon 7 in over 90 percent of cases while SMN2 variably exclude it; with dependence on cell type and environmental context. This splicing alteration is biologically significant as transcripts lacking exon 7 give rise to truncated and unstable proteins, which decreases the amount of functional SMN protein arising from the SMN2 locus.

SMA is caused by mutation or deletion of the SMN1 gene and retention of the SMN2 gene. The complete absence of SMN protein is embryonic lethal, but the presence of SMN2 in the absence of SMN1 in humans results in low levels of SMN protein, giving rise to SMA. Correlation between SMA severity and SMN protein expression has been illustrated both in SMA patients and in mouse models of SMA. Harada et al. Journal of Neurology, 249, 1211-19 (2002). SMN protein levels can vary widely and consequently, SMA is a disease of variable severity ranging from Type I, which is severe, with paralysis and death as early as birth, to Type IV, which is mild, with muscular weakness but no effect on life span. The most consistent correlative factor in this variability is the copy number of SMN2 genes (McAndrew et al., Am J Hum Genet., 60(6), 1411-22 (1997). The SMN2 gene locus is amplified in some individuals, with improved disease outcomes in those with higher copy numbers of SMN2. Furthermore, mouse models with more copy numbers of human SMN2 transgenes exhibited increased life span compared to littermates with fewer copy numbers (Hsieh-Li et al., Nat Genet., 24(1):66-70 (2000). Therefore, SMN protein levels are known to be directly correlated with positive disease outcomes (Lefebvre et al., Nat Genet., 16(3):265-9 (1997)), but for individuals with fewer copies of SMN2, it is critical to understand how splicing of this transcript may respond to more subtle cues, both for understanding the disease and also for developing therapeutics.

The invention is directed to methods of treating a subject in need of treatment. In some embodiments, the subject has been identified as being in need of treatment as a result of being diagnosed as having SMA. Symptom of SMA includes, but are not limited to: decreased expression of SMN exon 7 in a cell of the subject; decreased fetal movement; lethargy; loss or depression of muscular reflexes; hand tremors; peripheral neuropathies; large amplitude, prolonged, polyphasic discharges on active muscle contraction as detected by EMG (electromyography); myopathies; muscular paralysis, muscular atrophy; walking gait; muscular weakness; myasthenia; hypertrophied muscle bundles; fat infiltration in muscle bundles; fibrosis in muscle bundles; necrosis in muscle bundles; muscular dystrophies; atrophy of muscle bundles; decreased diameter of muscle fibers in the tail, trunk, or limbs; loss of strength of the respiratory muscles resulting in a weak cough, weak cry (infants), accumulation of secretions in the lungs or throat, respiratory distress; a bell-shaped torso (caused by using only abdominal muscles for respiration), lower weight, and in the case of various non-human animals, shorter and enlarged tails; chronic necrosis of the tail tip; subcutaneous edema; and reduced furry coat hair.

In certain embodiments, the subject has been diagnosed with SMA type I, also known as Werdnig-Hoffmann disease, which has an onset from 0-6 months. In certain embodiments, the subject has been diagnosed with SMA type II, also known as Dubowitz disease, which has an onset from 6-18 months. In certain embodiments, the subject has been diagnosed with SMA type III, also known as Kugelberg-Welander disease, which has an onset after 18 months. In certain embodiments, the subject has been diagnosed with SMA type IV, which has adult onset.

In certain embodiments, the subject is diagnosed as having SMA in utero. In certain embodiments, the subject is diagnosed as having SMA within one week after birth. In certain embodiments, the subject is diagnosed as having SMA within one month of birth. In certain embodiments, the subject is diagnosed as having SMA by 3 months of age. In certain embodiments, the subject is diagnosed as having SMA by 6 months of age. In certain embodiments, the subject is diagnosed as having SMA by 1 year of age. In certain embodiments, the subject is diagnosed as having SMA between 1 and 2 years of age. In certain embodiments, the subject is diagnosed as having SMA between 1 and 15 years of age. In certain embodiments, the subject is diagnosed as having SMA when the subject is older than 15 years of age.

In certain embodiments, the subject is a fetus and the heat shock response is induced in utero. The heat shock response can be induced using a heat shock inducing agent, or conditions that induce a heat shock response. In certain embodiments, the heat shock response is induced when the subject is less than one week old, less than one month old, less than 3 months old, less than 6 months old, less than one year old, less than 2 years old, less than 15 years old, or when the subject is older than 15 years old.

### Heat Shock Inducing Agents

Heat shock response may be induced in the subject (e.g., a cell of the subject) by administering a therapeutically effective amount of a heat shock inducing agent. Examples of heat shock inducing agents include protein synthesis inhibitors such as puromycin or azetidine, Hsp90 inhibitors such as geldanamycin (DMAG17), radicicol, or 17-AAG, and proteasome inhibitors such as MG132, bortezomib, omuralide, antiprotealide, epoxomicin, eponemycin or lactacystin. In further embodiments, agent may also be a serine protease inhibitor such as 3,4-dichloroisocoumarin (DCIC), tosyl-L-lysine chloromethyl ketone (TLCK) and tosyl-L-phenylalanine chloromethyl ketone (TPCK), an inflammatory mediator such as a cyclopentenone, prostaglandin, arachidonate, or phospholipase A₂, non-steroidal anti-inflammatory agent such as aspirin, ibuprofen and naproxen, a hydroxylamine derivative such as N-t-butyl hydroxylamine or bimoclomol, or a triterpenoid such as celastrol. Additional heart shock inducing agents include small molecule heat shock inhibitors such as quinacrine or 9-aminoacridine. In some embodiments, the heat shock response is induced using a pharmaceutical composition consisting essentially of a single heat shock inducing agent and one or more pharmaceutically acceptable carriers, in which the heat shock inducing agent is the active agent. In other embodiments, a plurality of heat shock inducing agents are administered.

In some embodiments, the heat shock inducing agent is a co-inducer. A disadvantage associated with some heat shock inducing agents is their lack of ability to target the heat shock response to the target cells (e.g., spinal motor neurons) and tendency to cause thermal injury. Co-inducers are able to overcome these disadvantages by inducing a heat shock response only in cells undergoing stress, such as degenerating motoneurons. Co-inducers therefore act to boost a heat stress response in cells where one is already occurring, rather than causing a heat stress response is all cells. Accordingly, in some embodiments, a co-inducer is also administered to the subject. Examples of co-inducers include non-steroidal anti-inflammatory drugs such as sodium salicylate, or indomethacin, or a hydroxylamine derivative such as bimoclomol or arimoclomol. The agent may also be a flavonoid such as quercetin or a benzylidene lactam compound such as KNK437. The agent may also be an arsenite compound or ethanol. In certain embodiments, the co-inducer is arimoclomol.

### Heat Shock Inducing Conditions

Heat shock response may also be induced in the cell with a heating means. The heating means may be photoelectric, mechanical, or chemical. The heating means may be capable of delivering heat to the cell noninvasively, such as transcutaneously. The heating means may also comprise a wire, lumen, receiver, probe or a catheter, as described in U.S. Pat. No. 8,486,127. The heating means may comprise optical fibers, a filament, or an implanted device. The heating means may be as described in U.S. Pat. No. 5,814,008, the contents of which are incorporated herein by reference.

The heating means may also comprise a sensor for measuring the temperature of a cell or tissue at a treatment site, and may produce a signal indicative of the temperature. The temperature may be measured with a thermocouple, a resistance temperature device, or a thermistor. The sensor may be a thermal needle sensor or a temperature probe. The temperature of the heating means may be controlled in response the signal. The temperature may be controlled by varying the amount of power supplied to energize the heat source to maintain the temperature at a predetermined level, or to prevent the temperature of the treated cell or tissue from exceeding a predetermined level.

The heating means may be a light emitting source such as an array of light emitting solid state devices. For example the light source may be a light emitting diode, electroluminescent device, laser, laser diode, vertical cavity emitting laser, or a filament lamp. The light may be of a specific wavelength, which may be a visible, near-infrared, or infrared wavelength. For example, the wavelength may be from 750 nm to 1 mm. The light source may be an intense laser, and may comprise using a two-photon method. The laser may comprise a highly collimated beam. The light source may also be a low power, non-coherent light source. The light may also be emitted at fewer particles per square meter (i.e., at a lower fluence rate) compared to a high intensity laser. For example, the fluence may be 30-25,000 Joules. The light intensity may be less than 500 mW/cm².

The heating means may also emit microwave or radio frequency energy, as described in U.S. Pat. Nos. 6,904,323 and 5,549,638, the contents of which are incorporated herein by reference. The heating means may comprise antenna, such as in an antenna array. The heating means may also comprise an ultrasound transducer.

The heating means may comprise a resistive element. The resistive element may be a resistive filament lamp. The heating means may also comprise a heated fluid or gas, which may be contained within a balloon, as described in U.S. Pat. Pub. No. 2007/0288075, the contents of which are incorporated herein by reference.

The heating means may comprise a chemical capable of increasing the internal temperature of the cell. For example, the chemical may comprise a dextran iron oxyhydroxide particle or iron complex. The chemical may also be gallium, indium, technetium, strontium, iodine, or other compound compatible with living tissue. The chemical may increase the internal temperature of the cell by increasing the rate of metabolism or oxidation of the chemical in the cell, such as by increasing blood oxygenation levels, as described in U.S. Pat. No. 4,569,836, the contents of which are incorporated herein by reference.

The effectiveness of treatment may be measured by evaluating the subject for a decrease in SMA symptoms in response to the administration of the heat shock activating agent or application of conditions to induce a heat shock response. Symptoms of SMA are described herein. For example, symptoms that can be used to evaluate the effectiveness of treatment include a decrease in hypotonia associated with absent reflexes; decreased fibrillation and muscle denervation as evaluated by electromyogram, or a decrease in serum creatine kinase levels.

Upon improvement of a patient's condition, a maintenance dose or level of a heat shock inducing agent or condition may be administered or provided, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms. For example, treatment of an infant with SMA can result in sufficient neural development for eventual reduction in the need to induce a heat shock response to increase expression of full length SMN2.

### Administration and Formulation of Heat Shock Inducing Agents

The present invention also provides pharmaceutical compositions that include heat shock inducing agents as an active ingredient, and a pharmaceutically acceptable liquid or solid carrier or carriers, in combination with the active ingredient. Any of the compounds described above as being suitable for the treatment of spinal muscular atrophy can be included in pharmaceutical compositions of the invention.

The heat shock inducing agents can be administered as pharmaceutically acceptable salts. Pharmaceutically acceptable salt refers to the relatively non-toxic, inorganic and organic acid addition salts of the heat shock inducing agents. These salts can be prepared in situ during the final isolation and purification of the compound, or by separately reacting a purified heat shock inducing agent with a suitable counterion, depending on the nature of the compound, and isolating the salt thus formed. Representative counterions include the chloride, bromide, nitrate, ammonium, sulfate, tosylate, phosphate, tartrate, ethylenediamine, and maleate salts, and the like. See for example Haynes et al., J. Pharm. Sci., 94, p. 2111-2120 (2005).

The pharmaceutical compositions includes one or more heat shock inducing agents together with one or more of a variety of physiological acceptable carriers for delivery to a patient, including a variety of diluents or excipients known to those of ordinary skill in the art. For example, for parenteral administration, isotonic saline is preferred. For topical administration, a cream, including a carrier such as dimethylsulfoxide (DMSO), or other agents typically found in topical creams that do not block or inhibit activity of the peptide, can be used. Other suitable carriers include, but are not limited to, alcohol, phosphate buffered saline, and other balanced salt solutions.

The formulations may be conveniently presented in unit dosage form and may be prepared by any of the methods well known in the art of pharmacy. Preferably, such methods include the step of bringing the active agent into association with a carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The methods of the invention include administering to a subject, preferably a mammal, and more preferably a human, the composition of the invention in an amount effective to produce the desired effect. The heat shock activating agents can be administered as a single dose or in multiple doses. Useful dosages of the active agents can be determined by comparing their in vitro activity and the in vivo activity in animal models. Methods for extrapolation of effective dosages in mice, and other animals, to humans are known in the art; for example, see U.S. Pat. No. 4,938,949.

The agents of the present invention are preferably formulated in pharmaceutical compositions and then, in accordance with the methods of the invention, administered to a subject, such as a human patient, in a variety of forms adapted to the chosen route of administration. The formulations include, but are not limited to, those suitable for oral, rectal, vaginal, topical, nasal, ophthalmic, or parental (including subcutaneous, intramuscular, intraperitoneal, intratumoral, intrathecal, and intravenous) administration.

In some embodiments, the heat shock inducing agent is administered into the central nervous system. For example, the heat shock inducing agent may be administered into the cerebrospinal fluid. Special techniques are known for delivering therapeutic agents to the central nervous system. See Begley, Pharmacology & Therapeutics 104, 29-45 (2004), for a review of methods delivery of therapeutic agents to the central nervous system.

Formulations of the present invention suitable for oral administration may be presented as discrete units such as tablets, troches, capsules, lozenges, wafers, or cachets, each containing a predetermined amount of the active agent as a powder or granules, as liposomes containing the celecoxib derivatives, or as a solution or suspension in an aqueous liquor or non-aqueous liquid such as a syrup, an elixir, an emulsion, or a draught. For example, in one embodiment, the heat shock activating agent is administered in drinking water. Such compositions and preparations typically contain at least about 0.1 wt-% of the active agent. The amount of heat shock activating agent (i.e., active agent) is such that the dosage level will be effective to produce the desired result in the subject.

Nasal spray formulations include purified aqueous solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier such as cocoa butter, or hydrogenated fats or hydrogenated fatty carboxylic acids. Ophthalmic formulations are prepared by a similar method to the nasal spray, except that the pH and isotonic factors are preferably adjusted to match that of the eye. Topical formulations include the active agent dissolved or suspended in one or more media such as mineral oil, petroleum, polyhydroxy alcohols, or other bases used for topical pharmaceutical formulations.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent may be incorporated into sustained-release preparations and devices.

The present invention is illustrated by the following example. It is to be understood that the particular examples, materials, amounts, and procedures are to be interpreted broadly in accordance with the scope and spirit of the invention as set forth herein.

### EXAMPLE

### Example 1: Heat Treatment Increases Full Length SMN Splicing, Illustrating Novel Therapeutic Targets for SMA

An important aspect of natural splicing manipulation occurs during development (especially evident in neuronal development) and also in response to environmental cues, such as cellular stress. One stress that was previously studied by the inventors is hypoxia. It was found that SMN2 is a hypoxia-responsive gene, whose splicing becomes altered under this condition, leading to increased skipping of exon 7 and decreased protein levels.

Here the inventors report that the induction of heat shock (41° for 8-24 hrs) improves the splicing of SMN2 by increasing the level of exon 7 inclusion in final transcripts. This leads to an increase in the functional SMN protein levels. We find that this splicing response is achieved through the upregulation of positive splicing factor, TRA2beta, and mediated though RNA cis element SE2. We report here that HSP90 function is essential for the splicing response upon heat treatment and can be prevented by either siRNA-mediated knock-down of HSP90 protein levels, or with the HSP90 inhibitor, 17-DMAG. Lastly, heat treatment was shown to not significantly affect SMA mice compared to no treatment controls.

### Results

Heat Shock Induces Full Length SMN Splicing and Increases SMN Protein.

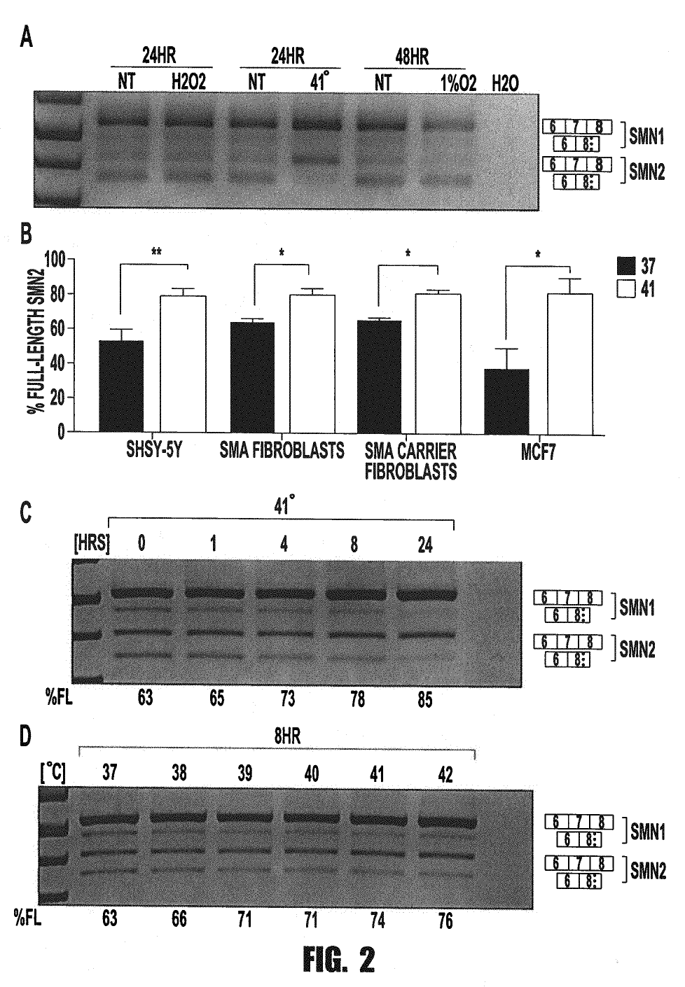

Environmental stresses have been shown to affect splicing and relate to many disease states. The inventors have previously shown that hypoxia leads to increased exon 7 exclusion in multiple cell lines. Bebee et al., Human Molecular Genetics, vol. 21, no. 19, pp. 4301-4313 (2012). However, to further their investigation in this area, the inventors wanted to investigate the response to hypoxia and other stresses in a neuronal-like cell line, and therefore chose to perform experiments in a differentiated neuroblastoma cell line (SH-SY5Y). To analyze the effect of environmental stresses on SMN2 splicing levels, three stresses were chosen that are related to neurodegenerative diseases and SMA in particular, to replicate in cell culture: oxidative stress, heat shock, and hypoxia. Cells were differentiated for 7 days in 5 μM all-trans retinoic acid before stress treatments. Cells were treated for oxidative stress (20 μM H₂O₂ for 24 hours), heat shock (41° C. for 24 hours), and hypoxia (1% O₂ for 48 hours) in separate experiments and total RNA was harvested following treatments (FIG. 2A). SMN transcripts were amplified by RT-PCR using primers that specifically recognize SMN exon 6 and exon 8. Because the sequence similarity of SMN1 and SMN2 precludes designing specific primers for discreet amplification of the isoforms, the inventors took advantage of a unique Dde1 restriction site in SMN2 exon 8, outside the coding region. DdeI enzyme digestion therefore differentiates SMN1 transcripts from SMN2 transcripts, by causing SMN2 to migrate faster than SMN1. Through this RT-PCR they found that both oxidative stress and hypoxia cause mild decreases in exon 7 inclusion (FIG. 2A). However, heat shock surprisingly caused a striking therapeutic increase in exon 7 inclusion. To determine if this response in conserved and common among cell lines, heat shock experiments were repeated in a disparate assortment of cell lines. Indeed, SMA patient fibroblasts (GM03813A; SMN1−/−), carrier fibroblasts (GM03814; SMN1+/−), and a breast cancer cells (MCF-7) all showed a splicing correction, or increase in the full-length, functional-producing form of SMN2, confirming that this phenomenon is conserved in all these cell lines. Furthermore, this phenomenon was found to be statistically significant in every cell line tested (FIG. 2B). Though all cell lines experienced a similar splicing alteration upon heat shock, differentiated SH-SY5Y cells had the most statistically significant change in splicing. Furthermore, as SMA is a disease of degenerating motor neurons, it is also most appropriate to have experiments based on a neuronal-derived cell line. Therefore, future experiments unless otherwise stated, are performed with this cell line.

Next, to determine the amount of timing and dosage responses of this corrective splicing response under heat shock, a time-course and variable heat intensity treatment was performed. The time-course was performed with 1, 4, 8, and 24 hours of 41° C. and 8 hrs dose response of increasing temperatures sequentially from 37° C. to 42° C. Time-course data reveals that the heat shock induced full-length SMN2 is time-dependent, with increases in SMN2 exon 7 inclusion peaking around 8 and 24 hrs (FIG. 2C). Similarly, this response is also dose-dependent with exon 7 inclusion sharply increasing with temperatures as low as 39° C. (FIG. 2D).

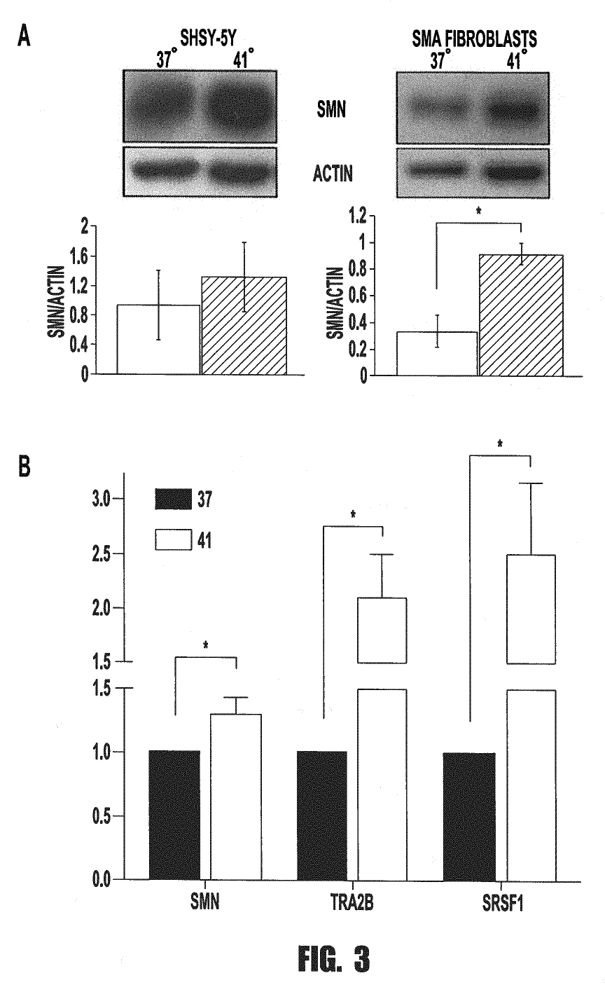

SMN2Δ7 splicing levels have been shown to correlate directly with decreased SMN protein levels. Lefebvre et al., Nat. Genet., vol. 16, no. 3, pp. 265-269 (1997). However, to verify under this stress condition that the alteration in RNA splicing levels correlates with increased SMN protein, whole cell lysate was harvested from SH-SY5Y and SMA patient fibroblast cells and treated with either 24 hrs of 37° C. or 41° C. Lysates were analyzed for protein levels through SDS-PAGE followed by western blotting. It was found SMN protein levels are upregulated in both cell types after heat shock. Representative Western blots of three trials are shown (FIG. 3). The levels of SMN protein are clearly upregulated in both cell lines after 41° C. treatments, but the difference is significant in SMA patient fibroblasts, as opposed to SHSY5Y cells. This is likely due to the fact that SH-SY5Y cells have an intact SAM gene while SMA patient fibroblasts do not. SMN1, which produces nearly all full-length protein, increases the pool of SMN protein, therefore changes in SMN protein are less obvious than in cells with a smaller SMN protein pool. Therefore, smaller improvements in SMN2 splicing in patient fibroblasts yield more obvious changes on the protein level.

Positive Splicing Factors and SMN are Upregulated Through mRNA Expression.

Regulation of protein levels is possibly due to increased transcription or stability of mRNA in addition to corrected splicing. Interestingly, the inventors found putative heat shock elements (HSEs) in SMN, TRA2beta, and SRSF1. Therefore, to understand the nature of the regulation of heat treatments on transcript levels, quantitative PCR (qPCR) analysis was performed on SMN, TRA2beta, and SRSF1 factors to determine transcript levels upon heat shock treatment. Differentiated SH-SY5Y cells were treated for 24 hrs with either 37° C. or 41° C., and then cells were harvested for total RNA. qPCR was performed with SYBR green reagent and normalized to GAPDH transcripts. All three transcripts were significantly upregulated with SMN at 1.3-fold, TRA2Beta at 2.2-fold, and SRSF1 at 2.6-fold increases upon heat treatment (FIG. 3B). This data indicates that both TRA2beta and SRSF1 are increased at the transcript level and that SMN is regulated not only on the splicing levels but also at the transcript level.

TRA2Beta Binding Sites are Critical for Heat Shock-Induced Exon 7 Inclusion

To determine if the upregulation of these two positive factors splicing factors is responsible for the increased SMN exon 7 inclusion, the inventors first tested the effect of mutating their respective binding sites. To study binding requirement, they utilized a minigene system, which is a small construct comprised of exon 6, a truncated intron 6 containing 5′ and 3′ ends of intron 6, and the entire sequence of exon 7, intron 7, and exon 8. This system allows us to manipulate binding sites in the minigene and then observe the resultant change in splicing by PCR amplifying using a T7 tag in exon 6.

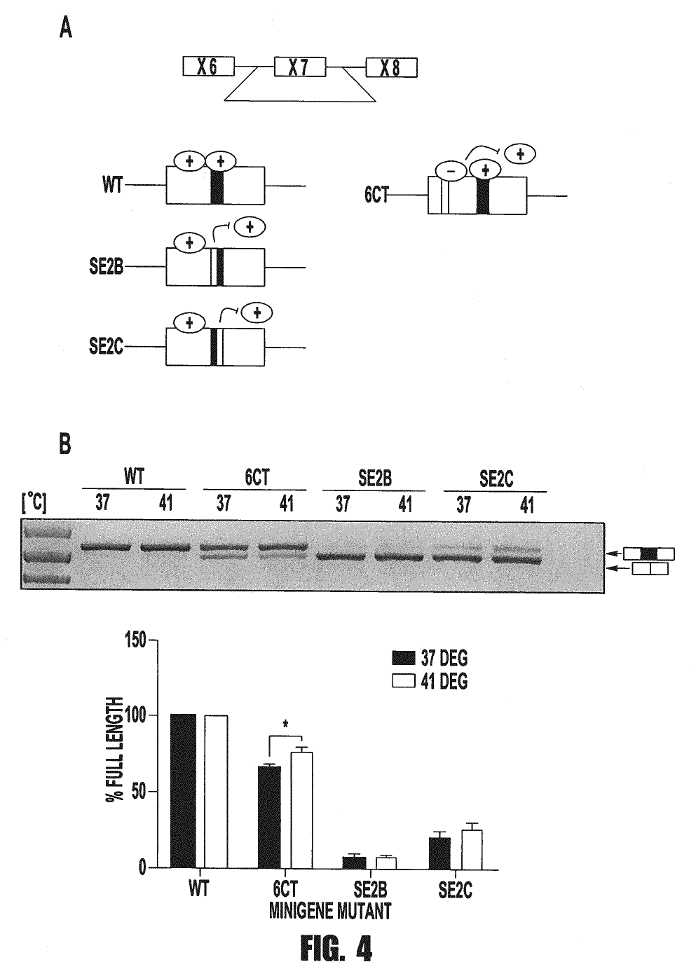

To investigate the necessity of binding regions, we mutated the parent “WT” minigene (or SMN1-like) into three separate mutant minigenes. The first mutant minigene “6ct” disrupts the SRSF1 binding site within exon 7 and creates an hnRNP A1 binding site (mimicking endogenous SMN2). The final two mutant minigenes “SE2b” and “SE2c” are both mutations of the 6 base pair long TRA2beta binding site in SMN exon 7. “SE2b” indicates a mutation in the first three nucleotides of the binding site to UUU and “SE2c” indicates a mutation in the last three nucleotides to UUU (model in FIG. 4A). Hofmann et al., Proc. Natl. Acad. Sci. U.S.A., vol. 97, no. 17, pp. 9618-9623 (2000).

The resultant minigenes were then nucleofected into differentiated SH-SY5Y cells 24 hours prior to heat treatment. Cells received 24 hours of 37° C. or 41° C., and then were harvested for total RNA. The 6ct mutant (mutant SRSF1 site) behaved like endogenous SMN2 with a reduction in full length SMN splicing compared to WT. Importantly, this minigene was heat shock responsive, increasing exon 7 inclusion significantly upon 41° C., indicating that even in the absence of this binding site, heat shock response was not abolished. Therefore, this SRSF1 binding site is not critical for the heat shock-induced full-length splicing. The SE2b and SE2c minigenes (mutant TRA2beta sites) decreased basal lull length splicing dramatically and furthermore, prevented an induction in exon 7 inclusion upon heat shock. Therefore, this supports an essential role for this TRA2beta binding site in exon 7 for the heat shock splicing correction as both the first half and second half of the binding region, are essential for the heat shock-induced full length SMN2 splicing (FIG. 4B).

TRA2beta Protein Levels are Necessary for Full-Length Splicing Under Heat Shock

Binding site analysis indicates that the role of SRSF1 may be less critical in heat shock regulation of SMN splicing than that of TRA2beta. To confirm these speculations, siRNA knock-downs of these key factors were performed. Due to difficulties in siRNA-mediated knock down of proteins in SH-SY5Y cells, we chose to perform these experiments in MCF-7 cells. These cells were chosen due to their knock down potential and due to their substantial and significant induction in full length splicing upon heat shock. siRNA duplexes for SRSF1 and for TRA2beta were transfected into MCF-7 cells 24 hrs before the 24 hour temperature treatment. Following siRNA and heat treatments, total RNA and protein lysates were harvested for RT-PCR and western blot analysis, respectively.

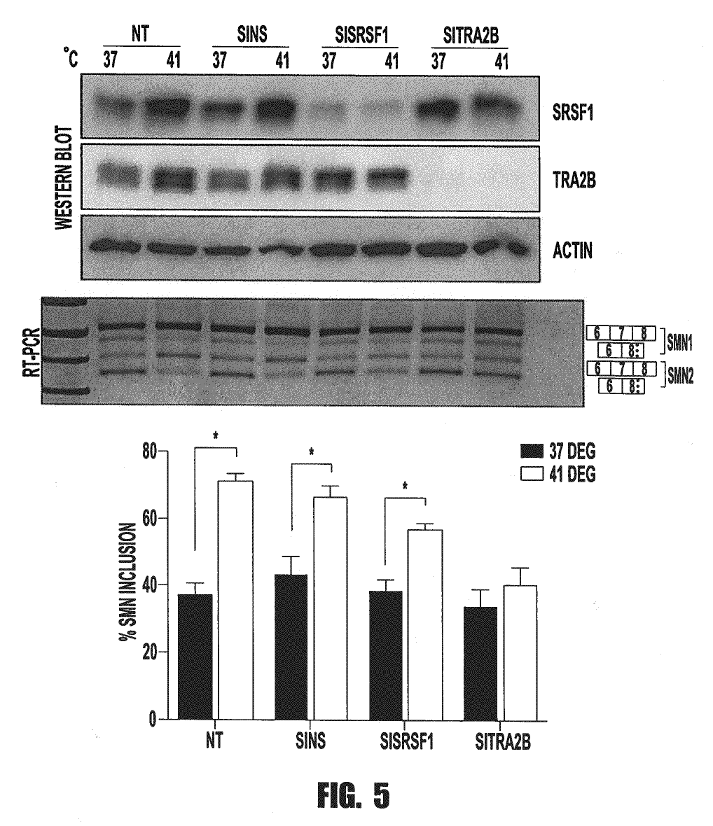

Western blots were analyzed for TRA2beta and SRSF1 protein levels, with beta actin as a loading control. No treatment (NT) group indicates non-transfection control and nonspecific siRNA (siNS) indicates siRNA transfection control. Comparison of these two groups indicates no significant changes in protein expression due to Transfection stress alone. siSRSF1 knock down group indicates an efficient knock down of SRSF1 protein levels and siTRA2beta knock-down group indicates an efficient knock down of TRA2beta protein levels (FIG. 5).

RT-PCR analysis of NT and siNS groups in SMN splicing levels shows no significant differences between these two groups. Both groups display an increase in full length splicing upon 41° C. treatment. The siSRSF1 group showed a diminished but still significant induction of full length splicing under 41° C. However, the siTRA2beta group shows a complete prevention of a full length splicing induction under 41° C. (FIG. 5). This data indicates that the protein levels of TRA2beta are important for the full length SMN splicing induced by heat shock, while SRSF1 protein levels are largely dispensable.

The Heat Shock Pathway Regulates SMN2 Splicing Through HSP90

Understanding how the SMN2 splicing correction is accomplished by players in the heat shock pathway is an important insight for this therapeutic response. The cellular heat shock response induces the large macro molecular structure, composed of heat shock proteins (HSPs) HSP90, HSP70, HSP40, and heat shock factor 1 (HSF1) to disassociate. After this dissociation occurs, HSF1 trimerizes and enters the nucleus as a transcription factor, inducing transcription of critical heat shock proteins. HSP90 has been implicated as a factor that contributes to several other neurodegenerative diseases (ALS (Kalmar et al., Journal of Neurochemistry, vol. 107, no. 2, pp. 339-350 (2008)), SBMA (Malik et al., Brain, vol. 136, no. 3, pp. 926-943 (2013)), retinitis pigmentosa (Parfitt et al., Cell Death and Disease, vol. 5, no. 5, p. e1236 (2014)). Therefore, HSP90 is of primary interest in our understanding of heat shock protein interactions in splicing of SMN2. Also of primary interest is HSF1, as the critical activator of the heat shock response.

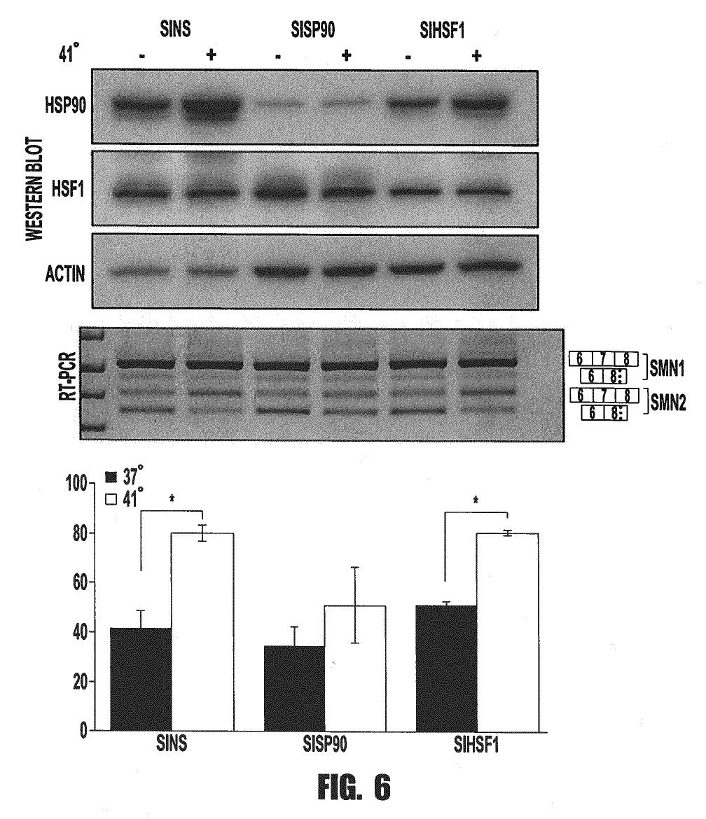

To begin investigation of whether these heat shock proteins play a role in SMN splicing under heat shock, siRNA knock-downs of these key factors was performed. As before, these experiments were carried out in MCF-7 cells. siRNA duplexes for HSP90 and for HSF1 were transfected into MCF-7 cells 24 hrs before the 24 hour temperature treatment. Following siRNA and heat treatments, total RNA and protein lysates were harvested for RT-PCR and western blot analysis, respectively. Western blots were analyzed for HSP90 and HSF1 with beta-actin as a loading control. Nonspecific siRNA (siNS) indicates siRNA transfection control. siHSP90 knock down group induced an efficient knock down of HSP90 protein levels, while siHSF1 knock down group induced only a partial knock down of HSF1 protein levels, but qPCR data indicated that HSF1 mRNA levels were decreased (FIG. 6). This data supports the fact that HSF1 is a very stable protein, and while siRNA is achieving targeted degradation of HSF1 mRNA, siRNA treatments must be extended, considering the rate of HSF1 protein turnover.

RT-PCR analysis of siNS group in SMN splicing levels shows an increase in full length splicing upon 41° C. treatment. The siHSP90 group showed a severely decreased full length splicing induction upon 41° C. treatment, removing any statistical induction (FIG. 5). However, considering the lack of protein knock down mentioned above the siHSF1 group, not surprisingly, shows no difference in SMN splicing from siNS group in response to heat shock treatment. Therefore, this data indicates that HSP90 is an important factor for inducing full length SMN2 splicing upon heat shock, while HSF1 protein levels remain to be more thoroughly investigated.

HSP90 Inhibitor, 17-DMAG, Prevents the Heat Shock-Induced Full Length SMN2 Splicing

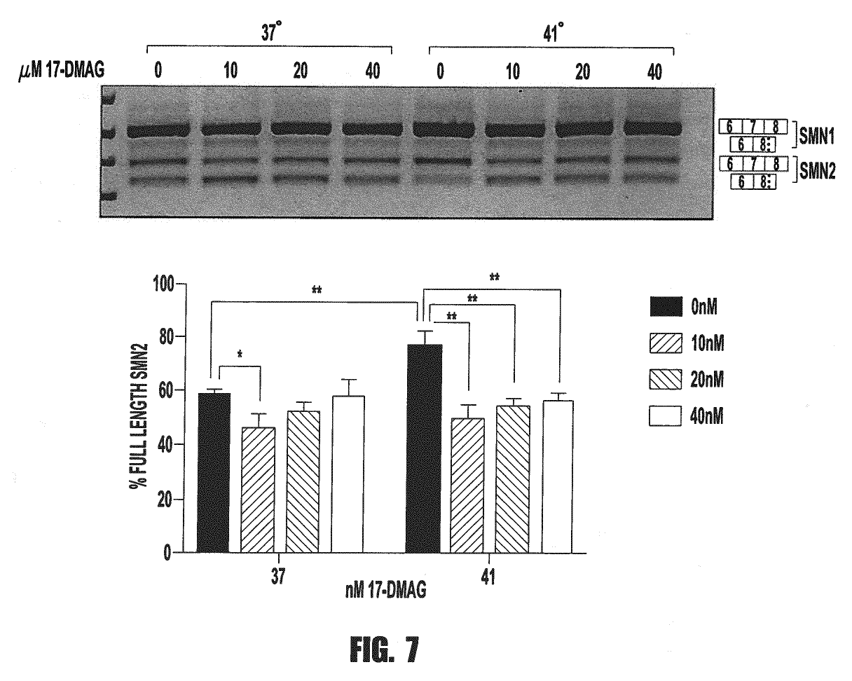

To validate the role of HSP90 in the SMN2 splicing under heat shock, the HSP90 ATPase inhibitor, 17-DMAG was administered to MCF-7 cells. 17-DMAG was administered at 0, 10 nM, 20 nM, and 40 nM concentrations the same time as the heat shock treatment, for the duration of 8 hours. Experiments were performed in parallel at 37° C. and 41° C. Treatments at 37° C. showed a significant decrease in SMN2 full length splicing only at the 10 nM dose, and at 20 nM and 40 nM concentrations the splicing levels began to approach basal splicing levels (0 μM). On the contrary, at 41° C., all three 17-DMAG doses, 10 nM, 20 nM, and 40 nM, significantly decreased the full length splicing to even below baseline splicing at 37° C. (FIG. 7). These data confirm HSP90 as a promoter of SMN2 full length splicing under heat shock. Full length SMN2 is quantitated for all samples and three trials are graphed, showing significant alterations in SMN splicing.

Transient, Daily Heat Treatments do not Significantly Improve SMA Mouse Body Weight or Full Length SMN2 Splicing

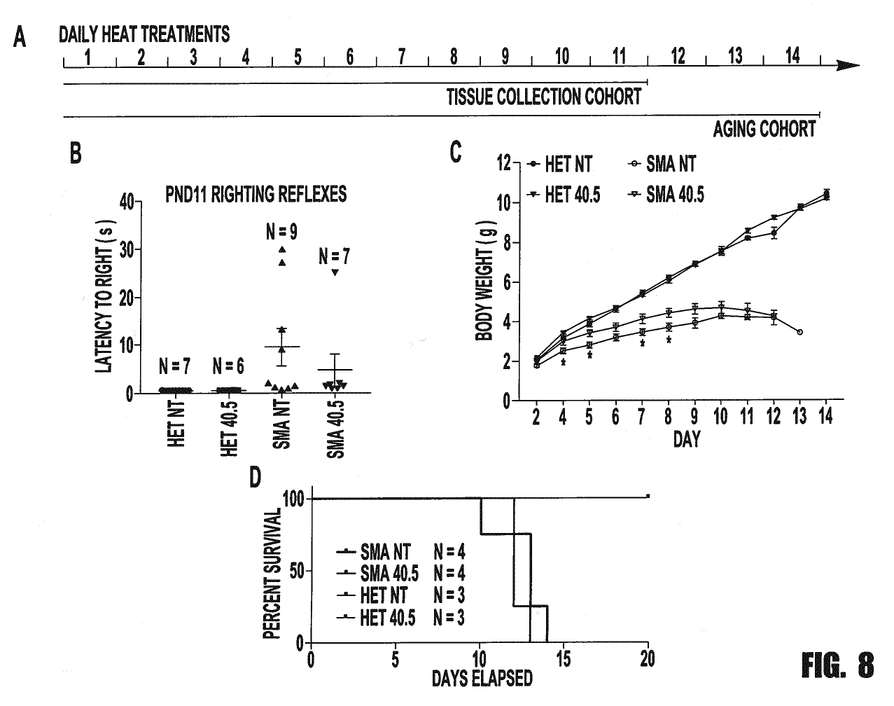

To determine if heat treatments can be a viable splicing modulatory technique in vivo, we performed transient, daily heat treatments in early postnatal severe SMA mouse pups. Beginning at PND1 the severe Taiwanese SMA mouse line was exposed to daily 40.5° C. treatment for 45 minutes in a temperature-controlled chamber. These treatment groups were compared to NT controls to determine if heat can affect severe SMA mouse phenotype. Two cohorts were designed; one extending only to 11 days, at which point mice were sacrificed and tissues harvested, and the other cohort extending treatments to end of life to assess changes in survival outcomes (FIG. 8A). Righting reflexes were measured to assess the level of muscle strength and coordination in untreated (NT) heterozygous (het) and SMA mice, and 40.5° treated het and SMA mice. Het righting reflexes in both treated and untreated groups averaged 0.5 seconds. SMA mouse righting reflexes on the other hand, improved the righting reflex trend with the heat treatment. However, due to large individual variability, sample sizes must be increased to determine statistical significance (FIG. 8B).

A second measure for increases in SMA disease outcome is bodyweight. SMA mice are characterized by decreased muscular strength, but also a decrease in body weight. To determine if bodyweight measures are changed upon heat treatment, we performed daily bodyweight measurements from day 2 to day 14. Heat treatments did not significantly increase SMA pup body weight compared to NT controls (FIG. 8C). This failure to increase body weight is also reflected in survival extension, with no significant extension in survival (FIG. 8D).

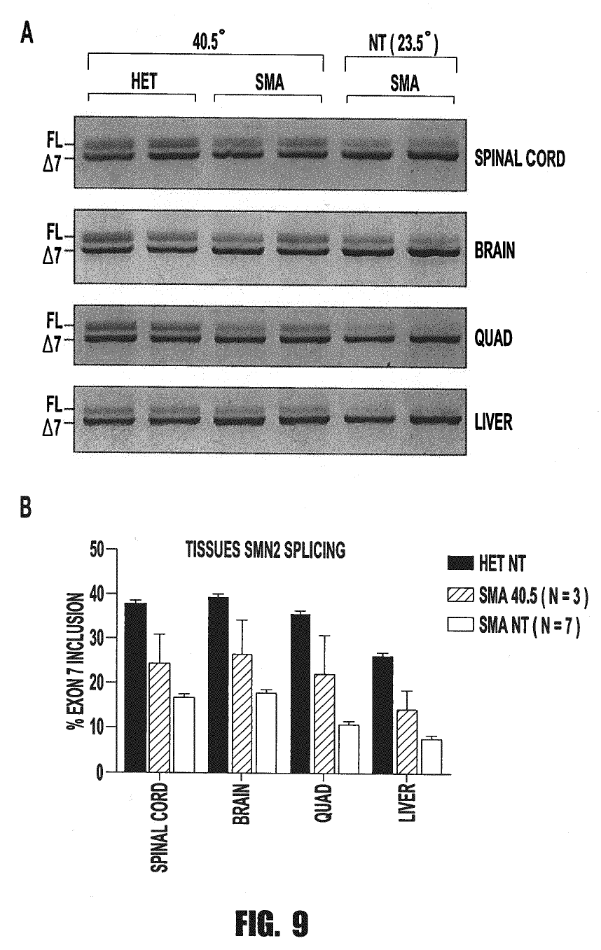

To ensure that the heat treatments are able to modulate splicing of SMN2 in vivo, tissue harvests and RNA extraction from brain, spinal cord, quad, and liver were performed. Semi-quantitative RT-PCR was used to determine splicing ratios in heterozygous mice, and heat-treated and NT SMA mice. RT-PCR splicing results of two representative mice from each group are shown in FIG. 9A. Quantitation of each treatment group is displayed. Heat treatment induces a trend toward full length splicing approaching that of heterozygous group however no significance was observed (FIG. 9B).

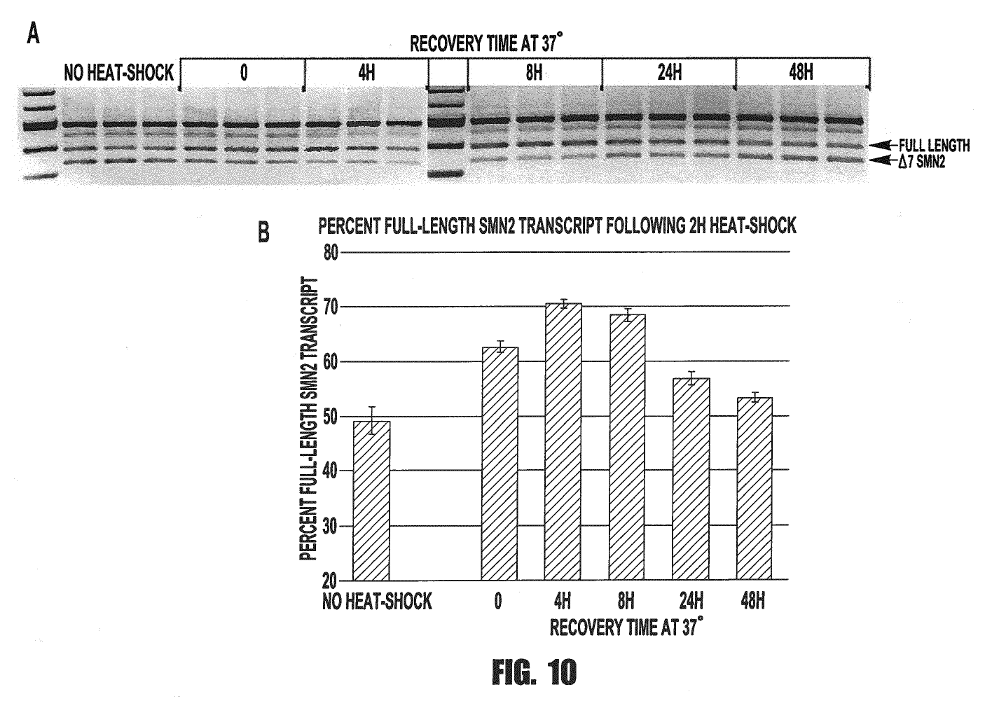

Additional experiments were carried out to determine the effect of heat-shock treatment and siRNA-mediated knock-down of HSF1. FIG. 10 shows that the increase in full-length SMN2 transcript in response to heat-shock treatment persists for at least 8 hours after a return to normal temperatures. These results are consistent with previous studies of the heat shock response in various species and cell types that show increased expression of HSPs for up to 24 hours after cells are returned to normal temperature. Fully characterizing the duration of the SMN2 splicing response to heat shock is crucial to developing effective therapeutic strategies that exploit components of the heat shock response to elevate SMN2 levels.

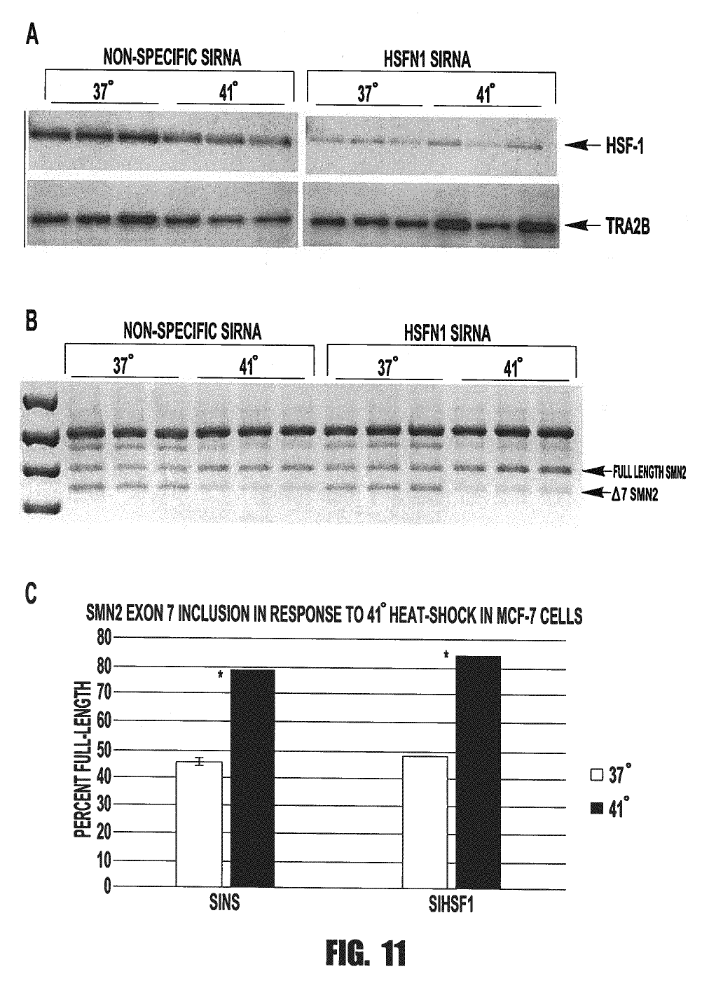

FIG. 11 shows that siRNA-mediated knock-down of HSF1 (heat-shock factor 1) levels does not affect the increased SMN2 exon 7 inclusion response to heat-shock. Unlike many of the heat shock response (HSR) proteins that function as chaperones that bind proteins to promote and maintain their proper folding, HSF1 is a transcription factor that is activated in response to cellular stress. One hypothesis to explain how heat shock treatment increases SMN2 exon 7 inclusion is that activated HSF1 either directly or indirectly promotes the increased expression of the splicing factors Tra2B and SRSF1 that was observed in response to heat shock (FIG. 3B). The fact that siRNA-mediated knock-down of HSF1 did not have a significant effect on increased SMN2 exon 7 inclusion argues against this model. While the siRNA-mediated knock down of HSF1 does not completely eliminate all HSF1 from the cells, and thus does not definitively rule out a role for HSF1, these data suggest that the splicing response does not require HSF1-mediated changes in gene expression. This, in turn, points toward a mechanism that involves the HSPs, such as HSP40, HSP70 or HSP90, the latter of which has already been implicated in the splicing response.

### DISCUSSION

These findings highlight a new aspect of heat shock regulation of splicing in general and SMN splicing in particular. Conventionally, it has been shown that heat shock stalls mRNA splicing and transcription of non-heat shock genes and halts these processes until the heat stress is removed or the cells acclimate. Shalgi et al., Cell Rep, vol. 7, no. 5, pp. 1362-1370 (2014). The inventors found that SMN exon 7 inclusion following splicing is improved and leads to greater amounts of SMN protein. Importantly, transcript levels of SMN, and two key splicing factors that promote inclusion of this exon, SRSF1, and TRA2beta, were both upregulated. This indicates that while not conventional heat shock proteins, all three of these factors, which are important for the proper splicing of the SMN transcripts, are highly regulated and promoted under heat shock stress conditions.

Interestingly, it was also found that the heat shock protein HSP90 affects the splicing of SMN exon 7. This is the first time that a heat shock protein has been implicated in the binding and splicing alteration of transcripts. HSP90 has been characterized as a DNA binding protein and has been shown to be capable of shuttling in and out of the nucleus, additionally, HSP90 is often recruited by viruses and has been shown to bind to the 3′UTR of certain viral genomic RNAs. Longshaw et al., Journal of cell science, vol. 117, no. 5, pp. 701-710 (2004). Therefore, a potential direct association between mRNA and HSP90 is plausible. HSP90 has a large variety of roles in the cell and is important for not only response to heat shock but also for housekeeping functions. The vast roles that HSP90 fills are largely dependent on the binding partners that it binds under different situations. A. Zuehlke and J. L. Johnson, Biopolymers, vol. 93, no. 3, pp. 211-217 (2010).

A key role for HSP90 in regulation of the heat shock response is to inactivate the heat shock transcription factor, HSF1. This is a negative feedback loop that maintains homeostasis and ensures that the heat shock response does not remain prolonged and that the cell can return to basal conditions following the stress. Studies in other neurodegenerative diseases such as amyotrophic lateral sclerosis, retinitis pigmentosa, and spinal and bulbar muscular atrophy, have found that inactivation of the HSP90 protein prolongs the heat shock response, which can promote neuronal survival. These studies focused on neurodegenerative disorders that involve the generation of toxic protein aggregations. In these studies it was found that the induction of heat shock response through HSP90 inactivation led to a decrease in these aggregates and was the primary mode of neuroprotection.

As SMA is not a disease that involves protein aggregates, the fact that the disease readout (as SMN splicing levels) did not improve upon HSP90 knock down or inhibition, indicates that motor neuron diseases may have different roles and needs for different heat shock components. Therefore, while other diseases conditions have improved upon decreasing HSP90 activity, the inventors hypothesize that SMA conditions could improve upon HSP90 activation. The field however, has very few options for inducting the activity of HSP90 and in these cases, the inducers are either impractical or too non-specific for administration into models of SMA or eventually patients. Benefits will be gained as the heat shock pathway becomes more clearly understood and with the advancement of small molecule screens, to find drugs that may increase the activity of HSP90.

Interestingly, in a population and clinical study in SMA patients, it was found that outcome in regions that experience cooler climates were improved compared to those in warmer climates, particularly with an extended period of ambulation. Bladen et al., J. Neurol., vol. 261, no. 1, pp. 152-163 (2014). These clinical correlations sparked the study of hypothermia treatment in a mouse model of SMA. Tsai et al., Human Molecular Genetics, vol. 25, no. 4, pp. 631-641 (2016). Following a hypothermia exposure, SMA pups displayed improved muscle morphology in the quadriceps, intercostal, and diaphragm muscles, as well as improved motor end plate occupancy in the quadriceps and additionally displayed body weight improvements compared to non-hypothermia-treated SMA mice. Hypothermia was achieved with exposure to crushed ice for 50 seconds daily, followed by a 5-minute recovery with a heat lamp.

Hypothermia treatment in this study showed potentially promising improvements in some SMA disease markers, however there is the possibility that the benefits are due to the warming period rather than the hypothermia period. For instance, the hypothermia treatment was 50 seconds in duration followed by 5 minutes of heating with a heat lamp. Under these conditions, the body temperature of the hypothermic pups was monitored, but the temperature delivered to the pups was not monitored or at least not reported. Therefore, the heat delivered to assist the mouse pups in reaching normal body temperature could have been a significant range of temperatures, with a significant possibility of delivering a stress response to the extremities in these pups. Furthermore, the 5 minutes of heat with a heat lamp administered to the pups, is a significantly longer treatment than the hypothermia treatment. An important control in this experiment would be to include a control group that received heat lamp treatment in the absence of hypothermia.

Furthermore, while cooler climates may have correlated with improved disease outcomes in SMA patients, there are also many cultural differences between countries, especially those with vastly different climates. Dietary differences, health care differences, and general activity level differences are all contributing factors that vary from region to region. Therefore, correlating these differences in SMA outcomes simply to temperature differences may not be taking all factors into consideration.

While the inventors were not able to show survival extension or statistical improvements in righting reflexes with their heat treatment paradigm, modifications can be implemented to troubleshoot a treatment plan. For instance, it is possible that under the current treatment plan, there is an initial improvement in condition but increasing heat stresses over time may inhibit this progress. A possible method to decrease the stress experienced by the mice could be decreasing the heat treatment from 45 minutes to 20 minutes to decrease the duration of the stress while maintaining the temperature to ensure splicing modification.

SMA is an ever-evolving field with great promise in clinical trials emerging. Chiriboga et al., Neurology, vol. 86, no. 10, pp. 890-897 (2016). However, once low levels of SMN are treated for these patients, it is imperative that backup therapies are available that can be used at combination therapies. There is a large variation in the way that patients respond to therapies, which can vary from complete recovery to a complete lack of response. Furthermore, the clinical trials, which are emerging as the most successful treatments, also involve invasive and time-sensitive techniques, with intrathecal injections, which are very effective and life-saving options, but they have limitations with efficiency of uptake and potential distribution problems. Furthermore, there has been data showing that peripheral tissues also require SMN restoration for full recovery in a mouse model of SMA. Hua et al., Nature, vol. 478, no. 7367, pp. 123-126, (2011). This leaves a need for these combination therapies, which would ideally be produced as an orally available option, with distribution to the rest of the body, outside the CNS, allowing for the whole body to be treated for low levels of SMN.

### Methods: Heat Shock

Mouse Treatment and Tissues Analysis

All experiments were performed according to institute standards and approved by the Institute Animal Care and Use Committee (IACUC) at The Research Institute at Nationwide Children's Hospital. Taiwanese mouse model was purchased as heterozygous carrier mice (Smn+/−; SMN2−/−) and transgene-carrying mice (Smn−/−; SMN2+/+) and were obtained from Jackson Laboratories (stock number #005058). These two genotypes were interbred to generate severe SMA mice (Smn−/−; SMN2+/−) and non-SMA control littermates (Smn+/−; SMN2+/−). Mother and pups were housed under normal or heat-treated conditions (40.5° daily for 45 minutes). starting at PND1. Weights were collected daily before the heat treatment (5:00 PM) and motor function was measured by the righting reflex assay on PND11. Pups were placed on their backs and time to rotating onto their ventral side was measured for up to 30 sec, pups were evaluated five times or up to three failed attempts of 30 sec each. Motor function and body weight were graphed and analyzed by Log-Rank Test (GraphPad Prism). Tissue harvests from mice were performed on PND11 45 minutes after removal from the treatment chamber and tissues were snap frozen in liquid nitrogen. Total RNA was isolated from tissues by homogenization in TRIzol (Invitrogen) and purification by RNeasy (Qiagen). Total RNA was utilized to make cDNA using random hexamers (Roche), and semi-quantitative RT-PCR was performed using forward primer and reverse primer. PCR products of full-length: 685 bp and Δ7: 631 bp were run on a 2.5% agarose gel and splicing ratios were determined by densitometry (ImageQuant TL, GE Healthcare Life Sciences).

Cell Culture and Reagents

Cell lines were all cultured according to guidelines from the ATCC cell depository. All cells were grown in 10% fetal bovine serum (hyclone), 50 μg/ml pen/strep), and 0.5 mM L-glutamine unless otherwise noted. SH-SY5Y cells were grown in DMEM:F12 (Gibco) for propagation and with an additional 5 μM all-trans retinoic acid (ATRA)(Sigma-Aldrich) added for differentiation media. Differentiation was achieved with 7 days of 5 μM ATRA treatment. MCF-7 cells were grown in DMEM (Gibco). SMA patient GM03813 (SMN1−/−) and carrier fibroblasts GM03814 (SMN1+/−) were grown in DMEM with 15% FBS.

All cell culture experiments were seeded to ensure sub confluent growth at time of harvesting to reduce other stresses (overcrowding, nutrient deprivation, and medium acidification).

17-DMAG (17-Dimethylaminoethylamino-17-demethoxygeldanamycin) was purchased from Cayman Chemicals and resuspended in DMSO to a 20 mM stock. Then treatments were administered to cultured cells at a density of 75% in 6 well plates.

Heat Shock Treatments

Cells grown under normal conditions (37° C.) were grown in a 5% CO₂, humidified incubator. Cells grown under heat shock conditions (38°−41° C.) were cultured at 5% CO₂, humidified incubators set to respective temperatures and monitored for temperature fluctuations with calibrated digital thermometer. Cells were maintained at their temperature treatment for the duration of the time point and harvested for RNA or protein directly following treatment.

Transfection Protocols

All transfected cells were cultured in their respective growth media without antibiotics. Minigene delivery was achieved by nucleofection (Lonza). SH-SY5Y cells were differentiated beginning at 50-60% confluence in 10 cM plates for 7 days. At the end of 7 days cells were approaching 90% confluence. At 7 days post-differentiation, cells were trypsinized and 500,000 cells were plated into a well of a 6 well plate per treatment group. For this cell type, Lanza nucleofector kit V was used for nucleofection as dictated by the company. 24 hours after SMN minigene nucleofection, the cells were treated for 24 hrs with either 37° or 41°.

Due to difficulties achieving protein knock down in SH-SY5Y cells, the inventors chose to use MCF-7 cells for siRNA treatments. siRNA delivery was achieved using Lipofectamine RNAiMAX (Life Technologies). Cells were seeded at 1 million cells per well of a 6 well plate. Transfections of siRNA duplexes was performed for a duration of 24 hrs and followed by 24 hrs of 37° or 41° without changing or removing media.

Minigene Production

SMNx-wt and SMNx-6ct, minigenes were provided by Dr. Luca Cartegni. All minigenes contain a C14U mutation in intron 7, which does not alter splicing ratios. Disruption of the TRA2B splice sites in mutations, SE2b and SE2c, were performed by site directed mutagenesis against SMNx-wt minigenes (Agilent Technologies).

All minigenes contain a T7 sequence and therefore, minigene-specific RT-PCR was performed using T7 forward primer and exon 8 reverse primer. RT-PCR (Sigma Taq Polymerase, Sigma-Aldrich) products for full-length (668 nt) and skipped (614 nt) were separated on a 2.5% agarose gel.

RT-PCR

Total RNA was isolated from samples using RNeasy kits (Qiagen). cDNA was created from RNA with Roche Transcriptor Reverse Transcriptase. cDNA was created from 1.5 ug of total RNA using random hexamers (Roche). PCR was performed with Sigma Taq Polymerase to detect exon 7 splicing of SMN1 and SMN2 transcripts. Primers were designed to flank exon 7 to visualize both full length and exon-skipped products: forward primer in exon 6 and reverse primer in exon 8. PCR cycles were performed with the following conditions: annealing temperature 62° C., 30 sec; 72° C. extension, 1 min; for 35 cycles. Importantly, there exists a unique digestion site (DdeI) within this PCR amplicon in exon 8 of SMN2, Therefore, to distinguish between SMN1 and SMN2 transcripts, PCR products were digested with the enzyme DdeI. Subsequent digested PCR products were then resolved on 2.5% agarose gels (product sizes: SMN1 full-length: 507 bp, SMN1Δ7: 453 bp, SMN2 full-length: 392 bp, SMN2Δ7: 338 bp). Splicing ratios were determined by densitometry (ImageQuant software, GE Healthcare Life Sciences) and statistical analysis (two tailed t test) performed using Graphpad Prism software.

Western Blot

Total protein lysates were isolated from tissue culture samples or mouse tissues in RIPA buffer. Protein samples were run on 10% SDS-PAGE gels. Transfers were performed overnight, 30V at 4° in Tris-Glycine buffer (BioRad) containing 20% methanol onto PVDF membranes. Membranes were probed for the following antibodies, all diluted in 5% milk: SMN (BD Transduction Laboratories, 1:1000), Beta-actin (clone AC-15, ThermoFisher 1:10,000), hnRNP A1 (Santa Cruz 1:5000), SAM68 (Santa Cruz 1:5000), SRSF1 (clone C-19, abeam 1:1000), TRA2Beta (abeam 1:1000), HSP90 (BD Transduction Laboratories, 1:1000), and HSF1 (Cell Signaling Technology, 1:1000). Quantitational analysis was performed using a BioRad Versa Doc scanner and quantified using Image Quant TL software (GE Healthcare Life Sciences).

Statistics

Splicing changes are graphically represented as averages of percent full length splicing (full length/full length+skipped) and standard deviation represented as error bars. Graphical representation and statistical analyses were performed using GraphPad Prism software. Statistical significance where identified by asterisks follow normal pvalue cutoffs: (*) p<0.05, (**) p<0.01, and (***) p<0.005.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of treating spinal muscular atrophy (SMA) comprising inducing a heat shock response in a subject in need thereof.

2. The method of claim 1, wherein the heat shock response is induced by heating the temperature of a tissue region of the subject above 37° C.

3. The method of claim 1, wherein the heat shock response is induced by administering a therapeutically effective amount of a heat shock inducing agent.

4. The method of claim 3, wherein administration of the heat shock inducing agent increases full length SMN2 mRNA and full length SMN2 protein in neural cells of the subject by at least about 50%.

5. The method of claim 3, wherein administration of the heat shock inducing agent increases full length SMN2 mRNA and full length SMN2 protein in muscle cells of the subject by at least about 50%.

6. The method of claim 3, wherein administration of the heat shock inducing agent increases full length SMN2 mRNA and full length SMN2 protein in glial cells of the subject by at least about 50%.

7. The method of claim 3, wherein the heat shock inducing agent is a protein synthesis inhibitor.

8. The method of claim 3, wherein the heat shock inducing agent is a proteasome inhibitor.

9. The method of claim 3, wherein the heat shock inducing agent is serine protease inhibitor.

10. The method of claim 3, wherein the heat shock inducing agent is an Hsp90 inhibitor.

11. The method of claim 3, wherein the heat shock inducing agent is a non-steroidal anti-inflammatory drug.

12. The method of claim 3, wherein the heat shock inducing agent is a hydroxylamine derivative.

13. The method of claim 3, wherein the heat shock inducing agent is a co-inducer.

14. The method of claim 13, wherein the co-inducer is arimoclomol.

15. The method of claim 3, wherein the heat shock inducing agent is administered into the central nervous system.

16. The method of claim 3, wherein the heat shock inducing agent is administered into the cerebrospinal fluid.

17. The method of claim 3, wherein the head shock inducing agent is administered into muscle.

18. The method of claim 3, wherein the heat shock inducing agent is administered in drinking water.

19. The method of claim 1, wherein the subject is a human.

20. The method of claim 1, wherein the subject is a fetus and the heat shock response is induced in utero.

21. The method of claim 1, wherein the subject has been diagnosed with type I SMA.

22. The method of claim 1, wherein the subject has been diagnosed with type II SMA.

23. The method of claim 1, wherein the subject has been diagnosed with type III SMA.

24. The method of claim 1, wherein the subject has been diagnosed with type IV SMA.
